(19) United States
(12) Patent Application Publication
(10) Pub. No.: US 2022/0331279 A1
(43) Pub. Date: Oct. 20, 2022

(54) THERAPEUTIC METHODS AND COMPOSITIONS FOR TREATING CANCER USING 6,8-BIS-BENZYLTHIO-OCTANOIC ACID AND A GLUTAMINASE INHIBITOR

(71) Applicant: Rafael Pharmaceuticals, lnc., Cranbury, NJ (US)

(72) Inventor: Sanjeev LUTHER, Cranbury, NJ (US)

(21) Appl. No.: 17/634,289

(22) PCT Filed: Aug. 14, 2020
(86) PCT No.: PCT/US2020/046296

§ 371 Date: Feb. 10, 2022

Related U.S. Application Data
(60) Provisional application No. 62/887,961, filed on Aug. 16, 2019.

(51) Int. Cl. A61K 31/20; A61K 31/501; A61P 35/00

(52) U.S. Cl. CPC A61K 31/20; A61K 31/501; A61P 35/00

(57) ABSTRACT

The invention provides methods and compositions for treating cancer by administering to a patient in need thereof a therapeutically effective amount of 6,8-bis-benzylthio-octanoic acid and a glutaminase inhibitor.

### CROSS-REFERENCE TO RELATED APPLICATIONS

This application is the national stage application of International (PCT) Patent Application Serial No. PCT/US2020/046296, filed Aug. 14, 2020, which claims the benefit of and priority to U.S. Provisional Patent Application Ser. No. 62/887,961, filed Aug. 16, 2019; the contents of each of which are hereby incorporated by reference.

### FIELD OF THE INVENTION

The invention provides methods and compositions for treating cancer by administration of 6,8-bis-benzylthio-octanoic acid and a glutaminase inhibitor.

### BACKGROUND

CPI-613 (6,8-bis-benzylthio-octanoic acid) is a first-in-class investigational small-molecule (lipoate analog), which targets the altered energy metabolism that is common to many cancer cells. CPI-613 has been evaluated in multiple phase I, I/II, and II clinical studies, and has been granted orphan drug designation for the treatment of pancreatic cancer, acute myeloid leukemia (AML), peripheral T-cell lymphoma (PTCL), Burkitt lymphoma and myelodysplastic syndromes (MDS).

A need exists to improve the safety and efficacy of treating cancer with CPI-613. The present invention addresses this need and provides other related advantages.

### SUMMARY

The invention provides methods and compositions for treating cancer in a human patient in need thereof by administering to the patient a therapeutically effective amount of (i) 6,8-bis-benzylthio-octanoic acid or a pharmaceutically acceptable salt thereof and (ii) a glutaminase inhibitor. The cancer may be, for example, relapsed or refractory. The cancer may be, for example, a lymphoma, leukemia, carcinoma, sarcoma, melanoma, myeloma, brain or spinal cord cancer, blastoma, germ cell tumor, cancer of the pancreas, colorectal cancer, myelodysplastic syndrome, or cancer of the prostate. In certain embodiments, the cancer is a lymphoma, leukemia, carcinoma, sarcoma, melanoma, or myeloma. In certain embodiments, the cancer is relapsed or refractory Hodgkin lymphoma, including relapsed or refractory Hodgkin lymphoma in a patient who has failed brentuximab vedotin and a PD-1 inhibitor, relapsed or refractory T-cell non-Hodgkin lymphoma, relapsed or refractory Burkitt's lymphoma, or high-grade B-cell lymphoma with rearrangements of MYC and BCL2 and/or BCL6. In certain embodiments, the glutaminase inhibitor is telaglenastat or a pharmaceutically acceptable salt thereof.

Another aspect of the invention provides a pharmaceutical composition comprising (i) 6,8-bis-benzylthio-octanoic acid or a pharmaceutically acceptable salt thereof and (ii) a glutaminase inhibitor. In certain embodiments, the glutaminase inhibitor is telaglenastat or a pharmaceutically acceptable salt thereof.

The foregoing aspects of the invention are described in more detail, along with additional embodiments, in the detailed description below.

### BRIEF DESCRIPTION OF THE DRAWINGS

FIG. 1 depicts results of treating human pancreatic cancer cells (AsPC-1) in vitro with 6,8-bis-benzylthio-octanoic acid (CPI-613), telaglenastat (CB-839), and combinations of the two. All compound concentrations listed are micromolar. Results are provided as percent of viable cells vs. control.

FIG. 2 depicts results of treating human colon cancer cells (LoVo) in vitro with 6,8-bis-benzylthio-octanoic acid (CPI-613), telaglenastat (CB-839), and combinations of the two. All compound concentrations listed are micromolar. Results are provided as percent of viable cells vs. control.

### DETAILED DESCRIPTION

The invention provides methods and compositions for treating cancer in a human patient in need thereof by administering to the patient a therapeutically effective amount of (i) 6,8-bis-benzylthio-octanoic acid or a pharmaceutically acceptable salt thereof and (ii) a glutaminase inhibitor. The cancer may be, for example, relapsed or refractory. The cancer may be, for example, a lymphoma, leukemia, carcinoma, sarcoma, melanoma, myeloma, brain or spinal cord cancer, blastoma, germ cell tumor, cancer of the pancreas, colorectal cancer, myelodysplastic syndrome, or cancer of the prostate. The methods may be further characterized by, for example, the dosing frequency and/or amount of the 6,8-bis-benzylthio-octanoic acid or a pharmaceutically acceptable salt thereof and a glutaminase inhibitor. The practice of the present invention employs, unless otherwise indicated, conventional techniques of medicinal chemistry, pharmacology, and biochemistry. Various aspects of the invention are set forth below in sections; however, aspects of the invention described in one particular section are not to be limited to any particular section.

### I. DEFINITIONS

To facilitate an understanding of the present invention, a number of terms and phrases are defined below.

The terms “a,” “an” and “the” as used herein mean “one or more” and include the plural unless the context is inappropriate.

The term “6,8-bis-benzylthio-octanoic acid” refers to the compound known as CPI-613, having the chemical structure

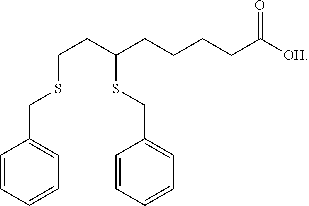 [SMILES: O=C(O)CCCCC(CCSCc1ccccc1)SCc1ccccc1]

The term “telaglenastat” refers to the compound known as CB-839, having the chemical structure

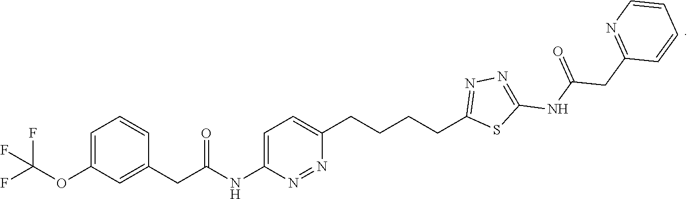 [SMILES: O=C(Cc1cccc(OC(F)(F)F)c1)Nc1ccc(CCCCc2nnc(NC(=O)Cc3ccccn3)s2)nn1]

Certain compounds contained in compositions of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

As used herein, the term “patient” refers to a human being in need of cancer treatment.

As used herein, the term “treating” includes any effect, e.g., lessening, reducing, modulating, ameliorating or eliminating, that results in the improvement, stabilization, or slowing progression of a condition, disease, disorder, or the like, or a symptom thereof. For example, treatment can include diminishment of a symptom of a disorder or complete eradication of a disorder. As another example, treatment can include slowing the progression of a disease, or preventing or delaying its recurrence, such as maintenance treatment to prevent or delay relapse.

“Therapeutically effective amount” refers to an amount of a compound sufficient to inhibit, halt, or cause an improvement in a disorder or condition being treated in a particular subject or subject population. For example, a therapeutically effective amount can be an amount of drug sufficient to slow the progression of a disease, or to prevent or delay its recurrence, such as maintenance treatment to prevent or delay relapse. In a human or other mammal, a therapeutically effective amount can be determined experimentally in a laboratory or clinical setting, or may be the amount required by the guidelines of the United States Food and Drug Administration, or equivalent foreign agency, for the particular disease and subject being treated. It should be appreciated that determination of proper dosage forms, dosage amounts, and routes of administration is within the level of ordinary skill in the pharmaceutical and medical arts.

As used herein, the term “pharmaceutical composition” refers to the combination of an active agent with an excipient, inert or active, making the composition suitable for administration to a human.

The phrase “pharmaceutically acceptable” is employed herein to refer to those compounds, materials, compositions, and/or dosage forms which are, within the scope of sound judgment, suitable for use in contact with the tissues of human beings with acceptable toxicity, irritation, allergic response, and other problems or complications commensurate with a reasonable benefit/risk ratio.

As used herein, the term “pharmaceutically acceptable excipient” refers to any of the standard pharmaceutical excipients suitable for use in humans. For examples of excipients, see e.g., Martin, Remington's Pharmaceutical Sciences, 15th Ed., Mack Publ. Co., Easton, Pa. [1975].

As used herein, the term “pharmaceutically acceptable salt” refers to any salt (e.g., acid or base) of a compound of the present invention which is suitable for administration to a human. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Further examples of salts include salts made using the ion pairing agents described in U.S. Pat. No. 8,263,653, the entire disclosure of which is incorporated by reference herein. Still further ion pairing agents can be selected with guidance from Handbook of Pharmaceutical Salts Properties, Selection and Use, UIPAC, Wiley-VCH, P. H. Stahl, ed., the entire disclosure of which is incorporated by reference herein.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited steps.

As a general matter, compositions specifying a percentage are by weight unless otherwise specified. Further, if a variable is not accompanied by a definition, then the previous definition of the variable controls.

### II. THERAPEUTIC APPLICATIONS

The invention provides methods and compositions for treating cancer in a human patient, comprising the step of administering to the human patient in need thereof a therapeutically effective amount of (i) 6,8-bis-benzylthio-octanoic or a pharmaceutically acceptable salt thereof and (ii) a glutaminase inhibitor. Accordingly, one aspect of the invention provides a method for treating cancer in a human patient. The method comprises administering to the human patient in need thereof a therapeutically effective amount of (i) 6,8-bis-benzylthio-octanoic acid or a pharmaceutically acceptable salt thereof and (ii) a glutaminase inhibitor.

### Type of Cancer

In certain embodiments, the cancer is associated with altered energy metabolism. As used herein, the term “cancer” is intended to include myelodysplastic syndromes. In certain embodiments of the present invention, the cancer is a myelodysplastic syndrome. In certain embodiments, the cancer is high risk myelodysplastic syndrome. In certain embodiments, the cancer is high risk myelodysplastic syndrome in a patient who has failed to respond, progressed, or relapsed while on hypomethylating therapy.

The method may be further characterized according to the severity or type of cancer. In certain embodiments, the cancer is Stage I or early stage cancer, in which the cancer is small and only in one area. In certain embodiments, the cancer is Stage II or III, in which the cancer is larger and has grown into nearby tissues or lymph nodes. In certain embodiments, the cancer is Stage IV or advanced or metastatic, in which the cancer has spread to other parts of the body.

In certain embodiments, the cancer is Stage I lymphoma, in which the cancer is found in one lymph node region or the cancer has invaded one extralymphatic organ or site but not any lymph node regions. In certain embodiments, the cancer is Stage II lymphoma, in which the cancer is found in two or more lymph node regions on the same side of the diaphragm or the cancer involves one organ and its regional lymph nodes, with or without cancer in other lymph node regions on the same side of the diaphragm. In certain embodiments, the cancer is Stage III lymphoma, in which there is cancer in lymph nodes on both sides of the diaphragm. In certain embodiments, the cancer is Stage IV lymphoma, in which the cancer has spread one or more organs beyond the lymph nodes.

In certain embodiments, the cancer is progressive or refractory. In certain embodiments, the cancer is metastatic. In certain embodiments, the cancer is recurrent or relapsed. In certain embodiments, the cancer is relapsed or refractory. In certain embodiments, the cancer is previously untreated. In certain embodiments, the cancer is previously untreated with systemic therapies. In certain embodiments, the cancer is previously untreated with systemic therapies or local treatment with chemoradiation. In certain embodiments, the patient has not received hematopoietic cell transplant. In certain embodiments, the patient has received hematopoietic cell transplant.

In certain embodiments, the cancer is a lymphoma, leukemia, carcinoma, sarcoma, melanoma, myeloma, brain or spinal cord cancer, blastoma, germ cell tumor, cancer of the pancreas, colorectal cancer, myelodysplastic syndrome, or cancer of the prostate. In certain embodiments, the cancer is a lymphoma, leukemia, carcinoma, sarcoma, melanoma, or myeloma.

In certain embodiments, the cancer is a lymphoma. In certain embodiments, the cancer is a T-cell lymphoma. In certain embodiments, the cancer is a B-cell lymphoma. In certain embodiments, the cancer is mantle cell lymphoma. In certain embodiments, the cancer is a leukemia. In certain embodiments, the cancer is an acute myeloid leukemia. In certain embodiments, the cancer is chronic myeloid leukemia. In certain embodiments, the cancer is acute lymphoblastic leukemia. In certain embodiments, the cancer is a carcinoma. In certain embodiments, the cancer is a sarcoma. In certain embodiments, the cancer is a myeloma. In certain embodiments, the cancer is a clear cell cancer. In certain embodiments, the cancer is a clear cell sarcoma. In certain embodiments, the cancer is a clear cell carcinoma. In certain embodiments, the cancer is a clear cell renal carcinoma. In certain embodiments, the cancer is a solid tumor. In certain embodiments, the cancer is a brain or spinal cord cancer. In certain embodiments, the cancer is a melanoma. In certain embodiments, the cancer is a blastoma. In certain embodiments, the cancer is a germ cell tumor.

In certain embodiments, the cancer is a cancer of the pancreas. In certain embodiments, the cancer is a metastatic pancreatic cancer. In certain embodiments, the cancer is a locally advanced pancreatic cancer. In certain embodiments, the cancer is a histologically or cytologically documented and measurable locally advanced pancreatic adenocarcinoma. In certain embodiments, the cancer is a histologically or cytologically documented and measurable metastatic pancreatic adenocarcinoma.

In certain embodiments, the cancer is a histologically or cytologically documented and measurable locally advanced pancreatic adenocarcinoma that is previously untreated. In certain embodiments, the cancer is a histologically or cytologically documented and measurable metastatic pancreatic adenocarcinoma that is previously untreated. In certain embodiments, the cancer is a histologically or cytologically documented and measurable locally advanced pancreatic adenocarcinoma that is previously untreated with systemic therapies. In certain embodiments, the cancer is a histologically or cytologically documented and measurable metastatic pancreatic adenocarcinoma that is previously untreated with systemic therapies. In certain embodiments, the cancer is a histologically or cytologically documented and measurable locally advanced pancreatic adenocarcinoma that is previously untreated with systemic therapies or local treatment with chemoradiation. In certain embodiments, the cancer is a histologically or cytologically documented and measurable metastatic pancreatic adenocarcinoma that is previously untreated with systemic therapies or local treatment with chemoradiation.

In certain embodiments, the cancer is a locally advanced pancreatic adenocarcinoma. In certain embodiments, the cancer is a metastatic pancreatic adenocarcinoma. In certain embodiments, the cancer is a locally advanced pancreatic adenocarcinoma that is previously untreated. In certain embodiments, the cancer is a metastatic pancreatic adenocarcinoma that is previously untreated. In certain embodiments, the cancer is a locally advanced pancreatic adenocarcinoma that is previously untreated with systemic therapies. In certain embodiments, the cancer is a metastatic pancreatic adenocarcinoma that is previously untreated with systemic therapies. In certain embodiments, the cancer is a locally advanced pancreatic adenocarcinoma that is previously untreated with systemic therapies or local treatment with chemoradiation. In certain embodiments, the cancer is a pancreatic adenocarcinoma that is previously untreated with systemic therapies or local treatment with chemoradiation.

In certain embodiments, the cancer is a cancer of the prostate. In certain embodiments, the cancer is a castration resistant prostate cancer. In certain embodiments, the cancer is a cancer of the lung. In certain embodiment, the cancer is non-small cell lung cancer. In certain embodiments, the cancer is a cancer of the colon. In certain embodiments, the cancer is a cancer of the rectum. In certain embodiments, the cancer is a colorectal cancer. In certain embodiments, the cancer is a cancer of the cervix. In certain embodiments, the cancer is a neuroendocrine tumor. In certain embodiments, the cancer is a gastroenteropancreatic neuroendocrine tumor. In certain embodiments, the cancer is a cancer of the liver. In certain embodiments, the cancer is a cancer of the uterus. In certain embodiments, the cancer is a cancer of the cervix. In certain embodiments, the cancer is a cancer of the bladder. In certain embodiments, the cancer is a cancer of the kidney. In certain embodiments, the cancer is a cancer of the breast. In certain embodiments, the cancer is a cancer of the ovary.

In certain embodiments, the cancer is Burkitt's lymphoma. In certain embodiments, the cancer is relapsed or refractory Burkitt's lymphoma. In certain embodiments, the cancer is relapsed or refractory Burkitt's lymphoma in which the patient has failed at least one previous line of therapy. In certain embodiments, the cancer is relapsed or refractory Burkitt's lymphoma in which the patient has failed prior bone marrow transplant. In certain embodiments, the cancer is double hit diffuse large B cell lymphoma. In certain embodiments, the cancer is high-grade B cell lymphoma with rearrangements of MYC and BCL2 and/or BCL6 (DHL/THL).

In certain embodiments, the cancer is Hodgkin lymphoma. In certain embodiments, the cancer is non-Hodgkin lymphoma. In certain embodiments, the cancer is T-cell non-Hodgkin lymphoma. In certain embodiments, the cancer is relapsed or refractory Hodgkin lymphoma. In certain embodiments, the cancer is relapsed or refractory non-Hodgkin lymphoma. In certain embodiments, the cancer is relapsed or refractory T-cell non-Hodgkin lymphoma.

In certain embodiments, the cancer is Hodgkin lymphoma in which the patient has not received hematopoietic cell transplant. In certain embodiments, the cancer is Hodgkin lymphoma in which the patient has received hematopoietic cell transplant. In certain embodiments, the cancer is non-Hodgkin lymphoma in which the patient has not received hematopoietic cell transplant. In certain embodiments, the cancer is non-Hodgkin lymphoma in which the patient has received hematopoietic cell transplant. In certain embodiments, the cancer is T-cell non-Hodgkin lymphoma in which the patient has not received hematopoietic cell transplant. In certain embodiments, the cancer is T-cell non-Hodgkin lymphoma in which the patient has received hematopoietic cell transplant. In certain embodiments, the cancer is relapsed or refractory Hodgkin lymphoma in which the patient has not received hematopoietic cell transplant. In certain embodiments, the cancer is relapsed or refractory Hodgkin lymphoma in which the patient has received hematopoietic cell transplant. In certain embodiments, the cancer is relapsed or refractory non-Hodgkin lymphoma in which the patient has not received hematopoietic cell transplant. In certain embodiments, the cancer is relapsed or refractory Hodgkin lymphoma in which the patient has or has not received hematopoietic cell transplant.

In certain embodiments, the cancer is relapsed or refractory Hodgkin lymphoma in which the patient has failed brentuximab vedotin and a PD-1 inhibitor. In certain embodiments, the cancer is relapsed or refractory Hodgkin lymphoma in which the patient has failed brentuximab vedotin and a PD-1 inhibitor and has received hematopoietic cell transplant. In certain embodiments, the cancer is relapsed or refractory Hodgkin lymphoma in which the patient has failed brentuximab vedotin and a PD-1 inhibitor and has not received hematopoietic cell transplant. In certain embodiments, the cancer is relapsed or refractory non-Hodgkin lymphoma in which the patient has received hematopoietic cell transplant. In certain embodiments, the cancer is relapsed or refractory T-cell non-Hodgkin lymphoma in which the patient has not received hematopoietic cell transplant. In certain embodiments, the cancer is relapsed or refractory T-cell non-Hodgkin lymphoma in which the patient has received hematopoietic cell transplant. In certain embodiments, the cancer is relapsed or refractory T-cell non-Hodgkin lymphoma in which the patient has or has not received hematopoietic cell transplant.

In certain embodiments, the cancer is a solid tumor or leukemia. In certain other embodiments, the cancer is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, esophagus cancer, liver cancer, an acoustic neuroma, oligodendroglioma, meningioma, neuroblastoma, or retinoblastoma. In certain other embodiments, the cancer is small cell lung cancer, non-small cell lung cancer, melanoma, cancer of the central nervous system tissue, brain cancer, Hodgkin's lymphoma, non-Hodgkin's lymphoma, cutaneous T-Cell lymphoma, cutaneous B-Cell lymphoma, or diffuse large B-Cell lymphoma. In certain other embodiments, the cancer is breast cancer, colon cancer, small-cell lung cancer, non-small cell lung cancer, prostate cancer, renal cancer, ovarian cancer, leukemia, melanoma, or cancer of the central nervous system tissue. In certain other embodiments, the cancer is colon cancer, small-cell lung cancer, non-small cell lung cancer, renal cancer, ovarian cancer, renal cancer, or melanoma.

### General Aspects of Administering a Therapeutic Agent to a Patient

Generally, the therapeutic agent—i.e., 6,8-bis-benzylthio-octanoic acid or a pharmaceutically acceptable salt thereof and a glutaminase inhibitor—is delivered to the patient in a therapeutically effective amount, sufficient to treat cancer. The treatment may involve one or several administrations on one or more days, and the dosage may be adjusted by the individual practitioner to achieve a desired effect. Preferably, the dosage amount of each agent should be sufficient to interact primarily with disease cells, leaving normal cells comparatively unharmed.

The dosage amount may be administered in a single dose or in the form of individual divided doses, such as one, two, three, or four times per day. In certain embodiments, the daily dosage amount is administered in a single dose. In the event that the response in a patient is insufficient at a certain dose, higher or more frequent doses may be employed to the extent of patient tolerance.

For the present combination therapy, each agent may be administered in a particular order and/or on the same or different days according to a treatment cycle. For example, a dose of 6,8-bis-benzylthio-octanoic acid may be administered to the patient prior to administering a glutaminase inhibitor, such as immediately prior, earlier in the day, or on an earlier day in a treatment cycle. In certain embodiments, the active agents may be administered on the same day of a treatment cycle, for example being co-administered simultaneously or one right after the other. In certain embodiments, a dose of a glutaminase inhibitor is administered to the patient prior to administering the 6,8-bis-benzylthio-octanoic acid, such as immediately prior, earlier in the day, or on an earlier day in a treatment cycle. In certain embodiments, treatment cycles may be repeated one or more times in order to maximize benefit to the patient.

### 6,8-Bis-Benzylthio-Octanoic Acid

6,8-Bis-benzylthio-octanoic acid may be administered in any suitable form, including as a solid or liquid, a free acid or salt. The 6,8-bis-benzylthio-octanoic acid may be crystalline, amorphous, or dissolved in solution. In certain embodiments, the 6,8-bis-benzylthio-octanoic acid is administered to the patient as a salt or ion pair. In certain embodiments, the 6,8-bis-benzylthio-octanoic acid is administered to the patient as a salt or ion pair with triethanolamine Exemplary ion pairing agents that may be used include, for example, a tertiary amine (such as triethylamine or triethanolamine), other amines such as diethylamine, diethanolamine, monoethanolamine, mefenamic acid and tromethamine, and combinations thereof. In certain embodiments, the ion pairing agent is an organic Bronsted base. In certain other embodiments, the ion pairing agent is an amine compound. In yet other embodiments, the ion pairing agent is a monoalkylamine, dialkylamine, trialkylamine, amino-substituted aliphatic alcohol, hydroxymonoalkylamine, hydroxydialkylamine, hydroxytrialkylamine, amino-substituted heteroaliphatic alcohol, alkyldiamine, substituted alkyldiamine, or optionally substituted heteroaryl group containing at least one ring nitrogen atom. In certain embodiments, the therapeutic agent is a salt of 6,8-bis-benzylthio-octanoic acid with an ion pairing agent selected with guidance from Berge et al., “Pharmaceutical Salts,” J. of Pharmaceutical Science, 1977; 66:1-19 or Handbook of Pharmaceutical Salts Properties, Selection and Use, IUPAC, Wiley-VCH, P. H. Stahl, ed., the entire disclosures of which are incorporated by reference herein. Ion pairing agents of particular note in the latter include, without limitation, those listed in Table 5, p. 342.

Additional exemplary ion pairing agents include, for example, polyethyleneimine, polyglutamic acid, ammonia, L-arginine, benethamine benzathine, betaine, calcium hydroxide, choline, deanol, diethanolamine(2,2′-iminobis(ethanol)), diethylamine, 2-(diethylamino)-ethanol, ethanolamine, ethylenediamine, N-methyl-glucamine, hydrabamine, 1H-imidazole, lysine, magnesium hydroxide, 4-(2-hydroxyethyl)-morpholine, piperazine, potassium hydroxide, 1-(2-hydroxyethyl)-pyrrolidine, sodium hydroxide, triethanolamine (2,2′,2″-nitrilotris(ethanol)), tromethamine, and zinc hydroxide. In certain other embodiments, the ion pairing agent is diisopropanolamine, 3-amino-1-propanol, meglumine, morpholine, pyridine, niacinamide, tris(hydroxymethyl)aminomethane, 2-((2-dimethylamino)ethoxy)ethanol, 2-(dimethylamino)ethanol, 1-(2-hydroxyethyl)pyrrolidine, or ammonium hydroxide. In certain other embodiments, the ion pairing agent is an alkali metal hydroxide or alkaline earth metal hydroxide, such as, for example, cesium hydroxide.

In certain embodiments, the 6,8-bis-benzylthio-octanoic acid has a purity of at least about 50% (w/w). In certain embodiments, the 6,8-bis-benzylthio-octanoic acid has a purity of at least about 60% (w/w). In certain embodiments, the 6,8-bis-benzylthio-octanoic acid has a purity of at least about 70% (w/w). In certain embodiments, the 6,8-bis-benzylthio-octanoic acid has a purity of at least about 80% (w/w). In certain embodiments, the 6,8-bis-benzylthio-octanoic acid has a purity of at least about 90% (w/w). In certain embodiments, the 6,8-bis-benzylthio-octanoic acid has a purity of at least about 95% (w/w). In certain embodiments, the 6,8-bis-benzylthio-octanoic acid has a purity of at least about 96% (w/w). In certain embodiments, the 6,8-bis-benzylthio-octanoic acid has a purity of at least about 97% (w/w). In certain embodiments, the 6,8-bis-benzylthio-octanoic acid has a purity of at least about 98% (w/w). In certain embodiments, the 6,8-bis-benzylthio-octanoic acid has a purity of at least about 99% (w/w).

### Glutaminase Inhibitor

The glutaminase inhibitor may be administered in any suitable form, including as a solid or liquid, a free acid or salt. The glutaminase inhibitor may be crystalline, amorphous, or dissolved in solution. In certain embodiments, the glutaminase inhibitor is administered to the patient as a salt or ion pair. When the glutaminase inhibitor is a basic compound, such as telaglenastat, it may be administered as an ion pair with an inorganic or organic acid. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. In certain embodiments, the therapeutic agent is a salt of a glutaminase inhibitor with an ion pairing agent selected with guidance from Berge et al., “Pharmaceutical Salts,” J. of Pharmaceutical Science, 1977; 66:1-19 or Handbook of Pharmaceutical Salts Properties, Selection and Use, IUPAC, Wiley-VCH, P. H. Stahl, ed., the entire disclosures of which are incorporated by reference herein. Ion pairing agents of particular note in the latter include, without limitation, those listed in Table 5, p. 342.

Any suitable glutaminase inhibitor may be used. In certain embodiments, the glutaminase inhibitor is telaglenastat or a pharmaceutically acceptable salt thereof. In certain embodiments, the glutaminase inhibitor is telaglenastat. In certain embodiments, the glutaminase inhibitor is telaglenastat hydrochloride.

### Route of Administration

The 6,8-bis-benzylthio-octanoic acid and glutaminase inhibitor may be administered to the patient by any suitable route. For example, in certain embodiments, the 6,8-bis-benzylthio-octanoic acid and/or glutaminase inhibitor is administered orally to the patient. In certain embodiments, the 6,8-bis-benzylthio-octanoic acid and glutaminase inhibitor are administered orally to the patient. In certain embodiments, the 6,8-bis-benzylthio-octanoic acid is administered orally to the patient. In certain embodiments, the glutaminase inhibitor is administered orally to the patient. In certain embodiments, the 6,8-bis-benzylthio-octanoic acid and/or glutaminase inhibitor is administered subcutaneously to the patient. In certain embodiments, the 6,8-bis-benzylthio-octanoic acid and/or glutaminase inhibitor is administered intravenously to the patient. In certain embodiments, the 6,8-bis-benzylthio-octanoic acid is administered as an IV infusion over two hours. In certain embodiments, the 6,8-bis-benzylthio-octanoic acid is administered as an IV infusion over two hours via a central venous catheter.

An advantage of oral dosing of the 6,8-bis-benzylthio-octanoic acid is that it permits substantially increased dosing flexibility as compared to IV. In the literature, 6,8-bis-benzylthio-octanoic acid has been reported as formulated as a 50 mg/mL solution in 1 M (150 mg/mL) aqueous triethanolamine, which is diluted from 50 mg/mL to as low as 4 mg/mL (e.g., 12.5 mg/mL) with sterile 5% dextrose for injection (D5W) prior to administration as an IV infusion over 30-120 minutes via a central venous catheter. Such an infusion is inconvenient for patients and effectively precludes regimens involving frequent and/or prolonged dosing. Since the half-life of 6,8-bis-benzylthio-octanoic acid after IV dosing is only about 1-2 hours (Pardee, T. S. et al., Clin. Cancer Res. 2014, 20, 5255-64), more frequent and/or prolonged dosing could advantageously be used to increase the patient's exposure to the drug.

For example, a possible IV schedule for the treatment of high risk MDS involves administering telaglenastat (e.g., from about 600 mg to about 1,200 mg) orally on each day of a 28 day cycle, followed each day by 6,8-bis-benzylthio-octanoic acid (e.g., about 2,000 mg/m²) IV. If administered orally, the practitioner would have more flexibility with respect to the 6,8-bis-benzylthio-octanoic acid dose and schedule. The 6,8-bis-benzylthio-octanoic acid could be orally administered in a single daily dose on days 1-5 of a 28 day cycle as in the IV schedule. Alternatively, the 6,8-bis-benzylthio-octanoic acid could be administered in two or more (e.g., three, four, or five) divided doses on days 1-5 of a 28 day cycle and/or the 6,8-bis-benzylthio-octanoic acid could be administered on fewer or additional days of the cycle, up to and including every day.

Another advantage of oral dosing is that it makes maintenance therapy feasible. For example, a patient who is treated successfully with first line therapy—with or without 6,8-bis-benzylthio-octanoic acid—and whose cancer is in partial or complete remission, may be treated orally with 6,8-bis-benzylthio-octanoic acid and a glutaminase inhibitor (e.g., telaglenastat) on a chronic basis in order to delay or prevent recurrence. The maintenance treatment may involve, for example, one, two, three, four, or five doses per day of the 6,8-bis-benzylthio-octanoic acid and glutaminase inhibitor on a regular basis, such as daily or weekly.

### Pharmaceutical Composition

One aspect of the invention provides a pharmaceutical composition comprising (i) 6,8-bis-benzylthio-octanoic acid or a pharmaceutically acceptable salt thereof and (ii) a glutaminase inhibitor. In certain embodiments, the glutaminase inhibitor is telaglenastat or a pharmaceutically acceptable salt thereof. In certain embodiments, the glutaminase inhibitor is telaglenastat. In certain embodiments, the glutaminase inhibitor is telaglenastat hydrochloride.

In the therapeutic methods of the invention, any suitable pharmaceutical composition may be used to administer the 6,8-bis-benzylthio-octanoic acid and the glutaminase inhibitor to the patient. The therapeutic agents may be administered together in the same pharmaceutical composition (e.g., fixed dose combination) or separately in different pharmaceutical compositions. In certain embodiments, one or more of the therapeutic agents is administered in a pharmaceutical composition that is a dry oral dosage form.

There is a wide variety of suitable formulations of pharmaceutical compositions of the present invention (see, e.g., Remington: The Science and Practice of Pharmacy, 20th ed., Gennaro et al. Eds., Lippincott Williams and Wilkins, 2000). In certain embodiments, the pharmaceutical composition is an oral dosage form chosen from tablet, pill, capsule, caplet, powder, granule, solution, suspension, and gel. Oral dosage forms may include pharmaceutically acceptable excipients, such as carriers, diluents, stabilizers, plasticizers, binders, glidants, disintegrants, bulking agents, lubricants, plasticizers, colorants, film formers, flavoring agents, preservatives, dosing vehicles, and any combination of any of the foregoing.

The pharmaceutical composition will generally include at least one inert excipient. Excipients include pharmaceutically compatible binding agents, lubricants, wetting agents, disintegrants, and the like. Tablets, pills, capsules, troches and the like can contain any of the following excipients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain a liquid excipient such as a fatty oil. In addition, dosage unit forms can contain various other materials that modify the physical form of the dosage unit, for example, coatings of sugar, shellac, or enteric agents. Further, a syrup may contain, in addition to the active compounds, sucrose as a sweetening agent and certain preservatives, dyes, colorings, and flavorings. In certain embodiments, the pharmaceutical composition comprises an excipient in an amount of about 5% to about 99%, such as about 10% to about 85%, by weight of the composition, with the therapeutic agent comprising the remainder. In certain embodiments, pharmaceutically acceptable excipients comprise about 20% to about 80% of the total weight of the composition. In certain embodiments, the pharmaceutical composition comprises the therapeutic agent in an amount of at least about 40% by weight of the composition, with one or more excipients comprising the remainder. In certain embodiments, the pharmaceutical composition comprises the therapeutic agent in an amount of at least about 50% by weight of the composition. In certain embodiments, the pharmaceutical composition comprises the therapeutic agent in an amount of at least about 60% by weight of the composition. In certain embodiments, the pharmaceutical composition comprises the therapeutic agent in an amount of at least about 70% by weight of the composition. In certain embodiments, the pharmaceutical composition comprises the therapeutic agent in an amount of at least about 80% by weight of the composition. In certain embodiments, the pharmaceutical composition comprises the therapeutic agent in an amount of at least about 90% by weight of the composition.

Diluents for solid (e.g., oral) compositions include, but are not limited to, microcrystalline cellulose (e.g. AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit), potassium chloride, powdered cellulose, sodium chloride, sorbitol and talc.

Binders for solid (e.g., oral) pharmaceutical compositions include, but are not limited to, acacia, tragacanth, sucrose, glucose, alginic acid, carbomer (e.g. Carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate and starch. In certain embodiments, the pharmaceutical composition comprises a binder in an amount of about 0.5% to about 25%, such as about 0.75% to about 15%, by weight of the composition. In certain embodiments, the pharmaceutical composition comprises a binder in an amount of about 1% to about 10% by weight of the composition.

The dissolution rate of a compacted solid pharmaceutical composition in a patient's stomach may be increased by the addition of a disintegrant to the composition. Disintegrants include, but are not limited to, alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, powdered cellulose, pregelatinized starch, sodium alginate, sodium starch glycolate (e.g. EXPLOTAB®) and starch. In certain embodiments, the pharmaceutical composition comprises a disintegrant in an amount of about 0.2% to about 30%, such as about 0.2% to about 10%, by weight of the composition. In certain embodiments, the pharmaceutical composition comprises a disintegrant in an amount of about 0.2% to about 5% by weight of the composition.

The pharmaceutical composition optionally comprises one or more pharmaceutically acceptable wetting agents. Such wetting agents are preferably selected to maintain the API in close association with water, a condition that is believed to improve bioavailability of the composition. Non-limiting examples of surfactants that can be used as wetting agents include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene, caprylic/capric mono- and diglycerides (e.g., Labrasol™ of Gattefosse), polyoxyethylene castor oil and polyoxyethylene hydrogenated castor oil; polyoxyethylene alkyl ethers, for example polyoxyethylene cetostearyl ether, polyoxyethylene fatty acid esters, for example polyoxyethylene stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefosse), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. In certain embodiments, the pharmaceutical composition comprises a wetting agent in an amount of about 0.25% to about 15%, such as about 0.4% to about 10%, by weight of the composition. In certain embodiments, the pharmaceutical composition comprises a wetting agent in an amount of about 0.5% to about 5% by weight of the composition. In certain embodiments, the pharmaceutical composition comprises a wetting agent that is an anionic surfactant. In certain embodiments, the pharmaceutical composition comprises sodium lauryl sulfate as a wetting agent. In certain embodiments, the pharmaceutical composition comprises sodium lauryl sulfate in an amount of about 0.25% to about 7%, such as about 0.4% to about 4%, by weight of the composition. In certain embodiments, the pharmaceutical composition comprises sodium lauryl sulfate in an amount of about 0.5% to about 2% by weight of the composition.

Lubricants (e.g., anti-adherents or glidants) can be added to improve the flow properties of solid compositions and/or to reduce friction between the composition and equipment during compression of tablet formulations. Excipients that may function as lubricants include, but are not limited to, colloidal silicon dioxide, magnesium trisilicate, powdered cellulose, starch, talc and tribasic calcium phosphate. Suitable lubricants further include glyceryl behapate (e.g., Compritol™ 888 of Gattefosse); stearic acid and salts thereof, including magnesium, calcium and sodium stearates; zinc stearate; glyceryl monostearate; glyceryl palmitostearate; hydrogenated castor oil; hydrogenated vegetable oils (e.g., Sterotex™ of Abitec); waxes; boric acid; sodium benzoate; sodium acetate; sodium stearyl fumarate; sodium fumarate; sodium chloride; DL-leucine; PEG (e.g., Carbowax™ 4000 and Carbowax™ 6000 of the Dow Chemical Company); sodium oleate; sodium lauryl sulfate; and magnesium lauryl sulfate. In certain embodiments, the pharmaceutical compositions comprises a lubricant in an amount of about 0.1% to about 10%, such as about 0.2% to about 8%, by weight of the composition. In certain embodiments, the pharmaceutical composition comprises a lubricant in an amount of about 0.25% to about 5% by weight of the composition. In certain embodiments, the pharmaceutical composition comprises magnesium stearate as a lubricant. In certain embodiments, the pharmaceutical composition comprises colloidal silicon dioxide. In certain embodiments, the pharmaceutical composition comprises talc. In certain embodiments, the composition comprises magnesium stearate or talc in an amount of about 0.5% to about 2% by weight of the composition.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid ethyl maltol, and tartaric acid.

Compositions may also be colored using any pharmaceutically acceptable colorant to improve their appearance and/or facilitate patient identification of the product and unit dosage level. The formulations of the invention may be buffered by the addition of suitable buffering agents.

In certain embodiments of the present invention, the therapeutic agent may be formulated as a pharmaceutically-acceptable oil; liposome; oil-water or lipid-oil-water emulsion or nanoemulsion; or liquid. To facilitate such formulations, the therapeutic agent may be combined with a pharmaceutically-acceptable excipient therefor.

As described in detail below, the pharmaceutical compositions may be specially formulated for administration in solid or liquid form, including those adapted for parenteral administration, for example, by subcutaneous, intramuscular, intravenous or epidural injection as, for example, a sterile solution or suspension, or sustained-release formulation.

Further examples of pharmaceutical formulations of 6,8-bis-benzylthio-octanoic acid are described in U.S. Pat. No. 8,263,653, the entire disclosure of which is incorporated by reference herein.

Methods of preparing pharmaceutical formulations or pharmaceutical compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

In certain embodiments, the pharmaceutical composition is a spray-dried dispersion. In certain embodiments, the pharmaceutical composition is a spray-dried dispersion comprising at least one polymer chosen from polyacrylate, polymethacrylate, poly(vinylpyrrolidone), hydroxypropyl methyl cellulose (HPMC), cellulose acetate phthalate (CAP), and hydroxypropyl methylcellulose acetate succinate (HPMCAS-M). In certain embodiments, the pharmaceutical composition is a spray-dried dispersion comprising at least one polymer chosen from Eudragit L100, poly(vinylpyrrolidone), hydroxypropyl methyl cellulose (HPMC), cellulose acetate phthalate (CAP), and hydroxypropyl methylcellulose acetate succinate (HPMCAS-M). In certain embodiments, the pharmaceutical composition is a spray-dried dispersion comprising at least one polymer chosen from Eudragit L100, poly(vinylpyrrolidone) viscosity grade K30 (PVP K30), hydroxypropyl methyl cellulose (HPMC), cellulose acetate phthalate (CAP), and hydroxypropyl methylcellulose acetate succinate (HPMCAS-M). In certain embodiments, the pharmaceutical composition is a spray-dried dispersion comprising at least one polymer chosen from Eudragit L100 and hydroxypropyl methylcellulose acetate succinate (HPMCAS-M). In certain embodiments, the pharmaceutical composition is a spray-dried dispersion comprising Eudragit L100. In certain embodiments, the pharmaceutical composition is a spray-dried dispersion comprising hydroxypropyl methylcellulose acetate succinate (HPMCAS-M).

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

In certain embodiments, one or more of the therapeutic agents are administered by intraparenteral administration. In certain other embodiments, one or more of the therapeutic agents are formulated for inhalational, oral, topical, transdermal, nasal, ocular, pulmonary, rectal, transmucosal, intravenous, intramuscular, subcutaneous, intraperitoneal, intrathoracic, intrapleural, intrauterine, intratumoral, or infusion methodologies or administration, or combinations of any thereof, in the form of aerosols, sprays, powders, gels, lotions, creams, suppositories, ointments, and the like. As indicated above, if such a formulation is desired, other additives known in the art may be included to impart the desired consistency and other properties to the formulation.

In certain embodiments, the pharmaceutical composition of the present invention is a unit dose composition. In certain embodiments, the pharmaceutical composition contains about 1 mg to about 5000 mg of the therapeutic agent. In certain embodiments, the pharmaceutical composition contains about 100 mg to about 3000 mg of the therapeutic agent. In certain embodiments, the pharmaceutical composition contains about 200 mg to about 2000 mg of the therapeutic agent. In certain embodiments, the pharmaceutical composition contains about 50 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2500 mg, or 3000 mg of therapeutic agent. In certain embodiments, the pharmaceutical composition contains about 300 mg, 500 mg, 700 mg, or 1000 mg of the therapeutic agent.

In certain embodiments, the pharmaceutical composition of the present invention comprises an emulsion, particle, or gel as described in U.S. Pat. No. 7,220,428. In certain embodiments, the pharmaceutical composition is a solid or liquid formulation having from about 0.1% to about 75% w/w lipids or fatty acid components. In certain embodiments, the formulation contains about 0.1% to about 15% w/v lipids and fatty acid components. In certain embodiments, the fatty acid component comprises saturated or unsaturated C4, C5, C6, C7, C8, C9, C10, C11, or C12 fatty acids and/or salts of such fatty acids. Lipids may include cholesterol and analogs thereof.

In certain embodiments, the pharmaceutical composition of 6,8-bis-benzylthio-octanoic acid comprises triethanolamine and 6,8-bis-benzylthio-octanoic acid in a mole ratio of triethanolamine to 6,8-bis-benzylthio-octanoic acid of about 10:1 to about 1:10. In certain embodiments, the mole ratio of triethanolamine to 6,8-bis-benzylthio-octanoic acid is about 10:1 to about 5:1. In certain embodiments, the mole ratio of triethanolamine to 6,8-bis-benzylthio-octanoic acid is about 8:1. In certain embodiments, the pharmaceutical composition comprises a 50 mg/mL solution of 6,8-bis-benzylthio-octanoic acid in 1M aqueous triethanolamine. In certain embodiments, the pharmaceutical composition comprises a solution of 6,8-bis-benzylthio-octanoic acid in 1M aqueous triethanolamine diluted from 50 mg/mL to as low as 12.5 mg/mL with sterile aqueous 5% dextrose for injection (D5W). In certain embodiments, the pharmaceutical composition comprises a solution of 6,8-bis-benzylthio-octanoic acid in 1M aqueous triethanolamine diluted from 50 mg/mL to about 12.5 mg/mL with sterile aqueous 5% dextrose for injection (D5W).

In certain embodiments, the pharmaceutical composition of the glutaminase inhibitor is an oral tablet comprising telaglenastat, such as in an amount of from about 150 mg to about 250 mg, about 250 mg to about 350 mg, about 350 mg to about 450 mg, about 450 mg to about 550 mg, about 550 mg to about 650 mg, about 650 mg to about 750 mg, or about 750 mg to about 850 mg.

### Dosing Amounts & Schedules

The 6,8-bis-benzylthio-octanoic acid and glutaminase inhibitor may be administered to the patient in any suitable dose according to any suitable schedule. The dose and schedule will vary based on the cancer being treated and can be readily determined by those of ordinary skill in the art in view of the 6,8-bis-benzylthio-octanoic acid and glutaminase inhibitor doses and schedules used in the prior art when administered alone or in combination with other agents, as well as the guidance provided herein. In certain embodiments, the dose is the maximum tolerated dose.

In certain embodiments, the 6,8-bis-benzylthio-octanoic acid is administered at a daily dose of about 150 mg/m² to about 3000 mg/m². In certain embodiments, the 6,8-bis-benzylthio-octanoic acid is administered at a daily dose of about 250 mg/m² to about 2500 mg/m². In certain embodiments, the first 6,8-bis-benzylthio-octanoic acid is administered at a daily dose of about 500 mg/m² to about 2000 mg/m². In certain embodiments, the 6,8-bis-benzylthio-octanoic acid is administered at a daily dose of about 150 mg/m². In certain embodiments, the 6,8-bis-benzylthio-octanoic acid is administered at a daily dose of about 200 mg/m². In certain embodiments, the 6,8-bis-benzylthio-octanoic acid is administered at a daily dose of about 250 mg/m². In certain embodiments, the 6,8-bis-benzylthio-octanoic acid is administered at a daily dose of about 300 mg/m². In certain embodiments, the 6,8-bis-benzylthio-octanoic acid is administered at a daily dose of about 350 mg/m². In certain embodiments, the 6,8-bis-benzylthio-octanoic acid is administered at a daily dose of about 400 mg/m². In certain embodiments, the 6,8-bis-benzylthio-octanoic acid is administered at a daily dose of about 450 mg/m². In certain embodiments, the 6,8-bis-benzylthio-octanoic acid is administered at a daily dose of about 500 mg/m². In certain embodiments, the 6,8-bis-benzylthio-octanoic acid is administered at a daily dose of about 600 mg/m². In certain embodiments, the 6,8-bis-benzylthio-octanoic acid is administered at a daily dose of about 700 mg/m². In certain embodiments, the 6,8-bis-benzylthio-octanoic acid is administered at a daily dose of about 800 mg/m². In certain embodiments, the 6,8-bis-benzylthio-octanoic acid is administered at a daily dose of about 900 mg/m². In certain embodiments, the 6,8-bis-benzylthio-octanoic acid is administered at a daily dose of about 1000 mg/m². In certain embodiments, the 6,8-bis-benzylthio-octanoic acid is administered at a daily dose of about 1100 mg/m². In certain embodiments, the 6,8-bis-benzylthio-octanoic acid is administered at a daily dose of about 1200 mg/m². In certain embodiments, the 6,8-bis-benzylthio-octanoic acid is administered at a daily dose of about 1300 mg/m². In certain embodiments, the 6,8-bis-benzylthio-octanoic acid is administered at a daily dose of about 1400 mg/m². In certain embodiments, the 6,8-bis-benzylthio-octanoic acid is administered at a daily dose of about 1500 mg/m². In certain embodiments, the 6,8-bis-benzylthio-octanoic acid is administered at a daily dose of about 1600 mg/m². In certain embodiments, the 6,8-bis-benzylthio-octanoic acid is administered at a daily dose of about 1700 mg/m². In certain embodiments, the 6,8-bis-benzylthio-octanoic acid is administered at a daily dose of about 1800 mg/m². In certain embodiments, the 6,8-bis-benzylthio-octanoic acid is administered at a daily dose of about 1900 mg/m². In certain embodiments, the 6,8-bis-benzylthio-octanoic acid is administered at a daily dose of about 2000 mg/m². In certain embodiments, the 6,8-bis-benzylthio-octanoic acid is administered at a daily dose of about 2500 mg/m². In certain embodiments, the 6,8-bis-benzylthio-octanoic acid is administered at a daily dose of about 3000 mg/m².

In certain embodiments, the 6,8-bis-benzylthio-octanoic acid is administered at a daily dose of about 1 mg to about 10,000 mg. In certain embodiments, the 6,8-bis-benzylthio-octanoic acid is administered at a daily dose of about 10 mg to about 7,500 mg. In certain embodiments, the 6,8-bis-benzylthio-octanoic acid is administered at a daily dose of about 100 mg to about 5,000 mg. In certain embodiments, the 6,8-bis-benzylthio-octanoic acid is administered at a daily dose of about 200 mg to about 4,000 mg. In certain embodiments, the 6,8-bis-benzylthio-octanoic acid is administered at a daily dose of about 300 mg to about 3,000 mg. In certain embodiments, the 6,8-bis-benzylthio-octanoic acid is administered at a daily dose of about 400 mg to about 2,500 mg. In certain embodiments, the 6,8-bis-benzylthio-octanoic acid is administered at a daily dose of about 500 mg to about 2,000 mg. In certain embodiments, the 6,8-bis-benzylthio-octanoic acid is administered at a daily dose of about 100 mg. In certain embodiments, the 6,8-bis-benzylthio-octanoic acid is administered at a daily dose of about 200 mg. In certain embodiments, the 6,8-bis-benzylthio-octanoic acid is administered at a daily dose of about 300 mg. In certain embodiments, the 6,8-bis-benzylthio-octanoic acid is administered at a daily dose of about 400 mg. In certain embodiments, the 6,8-bis-benzylthio-octanoic acid is administered at a daily dose of about 500 mg. In certain embodiments, the 6,8-bis-benzylthio-octanoic acid is administered at a daily dose of about 600 mg. In certain embodiments, the 6,8-bis-benzylthio-octanoic acid is administered at a daily dose of about 700 mg. In certain embodiments, the 6,8-bis-benzylthio-octanoic acid is administered at a daily dose of about 800 mg. In certain embodiments, the 6,8-bis-benzylthio-octanoic acid is administered at a daily dose of about 900 mg. In certain embodiments, the 6,8-bis-benzylthio-octanoic acid is administered at a daily dose of about 1,000 mg. In certain embodiments, the 6,8-bis-benzylthio-octanoic acid is administered at a daily dose of about 1,250 mg. In certain embodiments, the 6,8-bis-benzylthio-octanoic acid is administered at a daily dose of about 1,500 mg. In certain embodiments, the 6,8-bis-benzylthio-octanoic acid is administered at a daily dose of about 1,750 mg. In certain embodiments, the 6,8-bis-benzylthio-octanoic acid is administered at a daily dose of about 2,000 mg. In certain embodiments, the 6,8-bis-benzylthio-octanoic acid is administered at a daily dose of about 2,500 mg. In certain embodiments, the 6,8-bis-benzylthio-octanoic acid is administered at a daily dose of about 3,000 mg. In certain embodiments, the 6,8-bis-benzylthio-octanoic acid is administered at a daily dose of about 3,500 mg. In certain embodiments, the 6,8-bis-benzylthio-octanoic acid is administered at a daily dose of about 4,000 mg. In certain embodiments, the 6,8-bis-benzylthio-octanoic acid is administered at a daily dose of about 4,500 mg. In certain embodiments, the 6,8-bis-benzylthio-octanoic acid is administered at a daily dose of about 5,000 mg. In certain embodiments, the 6,8-bis-benzylthio-octanoic acid is administered at a daily dose of about 6,000 mg. In certain embodiments, the 6,8-bis-benzylthio-octanoic acid is administered at a daily dose of about 7,000 mg. In certain embodiments, the 6,8-bis-benzylthio-octanoic acid is administered at a daily dose of about 8,000 mg. In certain embodiments, the 6,8-bis-benzylthio-octanoic acid is administered at a daily dose of about 9,000 mg. In certain embodiments, the 6,8-bis-benzylthio-octanoic acid is administered at a daily dose of about 10,000 mg.

The daily dose of 6,8-bis-benzylthio-octanoic acid may be administered as one dose or divided into two or more doses—e.g., b.i.d. (two times a day), t.i.d. (three times a day), or q.i.d. (four times a day). In certain embodiments, the daily dose may be split into five doses administered in regular intervals during one day. At higher daily doses and/or when administered orally or subcutaneously, it will often be beneficial to administer the daily dose of 6,8-bis-benzylthio-octanoic acid b.i.d., t.i.d., or q.i.d. Since 6,8-bis-benzylthio-octanoic acid has a relatively short half life in the blood, splitting the daily dose may improve efficacy by prolonging exposure time and may also improve safety by reducing peak plasma concentration.

The 6,8-bis-benzylthio-octanoic acid may be administered pursuant to a treatment schedule that includes days in which a dose of 6,8-bis-benzylthio-octanoic acid is administered and days in which a dose of 6,8-bis-benzylthio-octanoic acid is not administered. For example, the 6,8-bis-benzylthio-octanoic acid may be administered pursuant to a schedule in which 6,8-bis-benzylthio-octanoic acid is administered during the early days of a cycle and then not administered during the latter portion of the cycle. In certain embodiments, the 6,8-bis-benzylthio-octanoic acid is administered on days 1-5 of a 28 day cycle. In certain embodiments, the 6,8-bis-benzylthio-octanoic acid is administered on days 1, 8, and 15 of a four week cycle. In certain embodiments, the 6,8-bis-benzylthio-octanoic acid is administered on days 1 and 3 of a two week cycle. In certain embodiments, the 6,8-bis-benzylthio-octanoic acid is administered on days 1-5 of a three week cycle. In certain embodiments, the 6,8-bis-benzylthio-octanoic acid is administered on days 1-5 of a two week cycle. In certain embodiments, the 6,8-bis-benzylthio-octanoic acid is administered on days 1-3 of a three week cycle. In certain embodiments, the 6,8-bis-benzylthio-octanoic acid is administered on days 1-3 of a two week cycle. In certain embodiments, the 6,8-bis-benzylthio-octanoic acid is administered every day. In certain embodiments, the 6,8-bis-benzylthio-octanoic acid is administered every other day. In certain embodiments, the 6,8-bis-benzylthio-octanoic acid is administered three days per week. In certain embodiments, the 6,8-bis-benzylthio-octanoic acid is administered two days per week. In certain embodiments, the 6,8-bis-benzylthio-octanoic acid is administered one day per week.

In certain embodiments, the glutaminase inhibitor (e.g., telaglenastat) is administered at a daily dose of from about 50 mg to about 2000 mg. In certain embodiments, the glutaminase inhibitor (e.g., telaglenastat) is administered at a daily dose of from about 50 mg to 150 mg, about 150 mg to about 250 mg, about 250 mg to about 350 mg, about 350 mg to about 450 mg, about 450 mg to about 550 mg, about 550 mg to about 650 mg, about 650 mg to about 750 mg, about 750 mg to about 850 mg, about 850 mg to about 950 mg, about 950 mg to about 1050 mg, about 1050 mg to about 1150 mg, about 1150 mg to about 1250 mg, about 1250 mg to about 1350 mg, about 1350 mg to about 1450 mg, about 1450 mg to about 1550 mg, about 1550 mg to about 1650 mg, about 1650 mg to about 1750 mg, about 1750 mg to about 1850 mg, or about 1850 mg to about 1950 mg. In certain embodiments, the glutaminase inhibitor (e.g., telaglenastat) is administered at a daily dose of from about 600 mg to about 800 mg. In certain embodiments, the glutaminase inhibitor (e.g., telaglenastat) is administered at a daily dose of from about 1200 mg to about 1600 mg. In certain embodiments, the glutaminase inhibitor (e.g., telaglenastat) is administered at a daily dose of about 600 mg. In certain embodiments, the glutaminase inhibitor (e.g., telaglenastat) is administered at a daily dose of about 800 mg. In certain embodiments, the glutaminase inhibitor (e.g., telaglenastat) is administered at a daily dose of about 1200 mg. In certain embodiments, the glutaminase inhibitor (e.g., telaglenastat) is administered at a daily dose of about 1600 mg.

The daily dose of glutaminase inhibitor may be administered as one dose or divided into two or more doses—e.g., b.i.d. In certain embodiments, the daily dose of glutaminase inhibitor is administered as one dose. In certain embodiments, the daily dose of glutaminase inhibitor is divided into two doses and administered b.i.d. In certain embodiments, the daily dose of telaglenastat is administered as one dose. In certain embodiments, the daily dose of telaglenastat is divided into two doses and administered b.i.d.

In certain embodiments, the dose of telaglenastat is administered orally. In certain embodiments, the dose of telaglenastat is administered orally without food. In certain embodiments, the dose of telaglenastat is administered orally with food.

For simplicity, the glutaminase inhibitor will typically be administered pursuant to a treatment cycle that is the same length as each treatment cycle for 6,8-bis-benzylthio-octanoic acid (e.g., 2 weeks, three weeks, four weeks, etc.). Like the cycle for 6,8-bis-benzylthio-octanoic acid, the glutaminase inhibitor cycle may include days in which a dose of glutaminase inhibitor is administered and days in which a dose of glutaminase inhibitor is not administered. For example, the glutaminase inhibitor may be administered pursuant to a schedule in which glutaminase inhibitor is administered on the same days that 6,8-bis-benzylthio-octanoic acid is administered, and is not administered on the days 6,8-bis-benzylthio-octanoic acid is not administered. Alternatively, the glutaminase inhibitor may be administered on some but not all days in which 6,8-bis-benzylthio-octanoic acid is administered, and/or may be administered on some but not all days on which 6,8-bis-benzylthio-octanoic acid is not administered. In certain embodiments, the glutaminase inhibitor may be administered on each day of the cycle.

In certain embodiments, the dosing cycle is repeated at least once. In certain embodiments, the method of the present invention comprises treatment with two cycles or more. In certain embodiments, the method of the present invention comprises treatment with three cycles or more. In certain embodiments, the method of the present invention comprises treatment with four cycles or more. In certain embodiments, the method of the present invention comprises treatment with five cycles or more. In certain embodiments, the method of the present invention comprises treatment with six cycles or more. In certain embodiments, the method of the present invention comprises treatment with seven cycles or more. In certain embodiments, the method of the present invention comprises treatment with eight cycles or more. In certain embodiments, the method of the present invention comprises treatment with nine cycles or more. In certain embodiments, the method of the present invention comprises treatment with ten cycles or more. In certain embodiments, the method of the present invention comprises regular treatment with 6,8-bis-benzylthio-octanoic acid and a glutaminase inhibitor, including on a daily or weekly basis, for an extended period of time, such as at least one month, six months, one year, two years, three years, or longer.

### Patients for Treatment

The therapeutic methods may be further characterized according to the patient to be treated. In the present invention, the patient is a human being. In certain embodiments, the patient is an adult. In certain embodiments, the patient is an adult at least 60 years of age. In certain embodiments, the patient is a child.

### Treatment Efficacy and Safety

The therapeutic method of the present invention may be further characterized by the efficacy and safety of the treatment. Preferably, the method provides an acceptable safety profile, with the benefit of treatment outweighing the risk. When tested in a phase II or phase III clinical trial of at least 10 patients with cancer, the method of the present invention preferably provides an overall response rate of at least about 10%, a duration of response of at least about 1 month, progression-free survival (PFS) of at least about 1 month, and/or overall survival (OS) of at least about 1 month. Preferably, the phase II or phase III clinical trial comprises at least 15 patients. More preferably, the phase II or phase III clinical trial comprises at least 20 patients. More preferably, the phase II or phase III clinical trial comprises at least 25 patients. More preferably, the phase II or phase III clinical trial comprises at least 50 patients. More preferably, the phase II or phase III clinical trial comprises at least 100 patients. More preferably, the phase II or phase III clinical trial comprises at least 200 patients. More preferably, the phase II or phase III clinical trial comprises at least 300 patients. More preferably, the phase II or phase III clinical trial comprises at least 400 patients. More preferably, the phase II or phase III clinical trial comprises at least 500 patients.

Preferably, the method of the present invention provides an overall response rate of at least about 20% in patients. More preferably, the method of the present invention provides an overall response rate of at least about 30%. More preferably, the method of the present invention provides an overall response rate of at least about 40%. More preferably, the method of the present invention provides an overall response rate of at least about 50%. More preferably, the method of the present invention provides an overall response rate of at least about 60%. More preferably, the method of the present invention provides an overall response rate of at least about 70%. More preferably, the method of the present invention provides an overall response rate of at least about 80%. More preferably, the method of the present invention provides an overall response rate of at least about 90%.

Preferably, the method of the present invention provides a duration of response, PFS, and/or OS of at least about 2 months. Preferably, the method of the present invention provides a duration of response, PFS, and/or OS of at least about 3 months. Preferably, the method of the present invention provides a duration of response, PFS, and/or OS of at least about 4 months. Preferably, the method of the present invention provides a duration of response, PFS, and/or OS of at least about 5 months. Preferably, the method of the present invention provides a duration of response, PFS, and/or OS of at least about 6 months. Preferably, the method of the present invention provides a duration of response, PFS, and/or OS of at least about 7 months. Preferably, the method of the present invention provides a duration of response, PFS, and/or OS of at least about 8 months. Preferably, the method of the present invention provides a duration of response, PFS, and/or OS of at least about 9 months. Preferably, the method of the present invention provides a duration of response, PFS, and/or OS of at least about 10 months. Preferably, the method of the present invention provides a duration of response, PFS, and/or OS of at least about 11 months. Preferably, the method of the present invention provides a duration of response, PFS, and/or OS of at least about 12 months. Preferably, the method of the present invention provides a duration of response, PFS, and/or OS of at least about 14 months. Preferably, the method of the present invention provides a duration of response, PFS, and/or OS of at least about 16 months. Preferably, the method of the present invention provides a duration of response, PFS, and/or OS of at least about 18 months. Preferably, the method of the present invention provides a duration of response, PFS, and/or OS of at least about 20 months. Preferably, the method of the present invention provides a duration of response, PFS, and/or OS of at least about 24 months.

In certain embodiments, the overall response rate, duration of response, and progression-free survival mentioned above are measured in a phase II clinical trial. In certain embodiments, the overall response rate, duration of response, and progression-free survival mentioned above are measured in a phase III clinical trial.

The description above describes multiple aspects and embodiments of the invention, including therapeutic applications, treatment methods, and pharmaceutical compositions. The patent application specifically contemplates all combinations and permutations of the aspects and embodiments.

### III. EXAMPLES

The invention now being generally described, will be more readily understood by reference to the following examples, which are included merely for purposes of illustration of certain aspects and embodiments of the present invention, and are not intended to limit the invention.

### Example 1—Activity of CPI-613, CB-839, and Combinations Thereof Towards Pancreatic and Colon Cancer Cells In Vitro

The ability of CPI-613, CB-839, and combinations thereof to kill pancreatic cancer cells and colon cancer cells in vitro was evaluated. Experimental procedures and results are provided below.

### Part I—Experimental Procedure

Human pancreatic cancer AsPC-1 cells and human colon cancer LoVo cells were obtained from American Type Cell Culture, Manassas, Va. All tumor cells were maintained at 37° C. in a humidified atmosphere with 5% CO₂ in T75 tissue culture flasks, containing 10 mL of DMEM (AsPC-1) or 10 mL of F12K (LoVo), each containing L-glutamine (2 mM), fetal bovine serum (FBS, 10%), penicillin (100 IU/mL) and streptomycin (100 μg/mL). The cells were split at a ratio of 1:5 every 4-5 days by using trypsin and were re-suspended in fresh media in new flasks as described above. Cells were harvested for experiments at 70-90% confluence.

Cancer cells were seeded (5,000 per well in 96-well plate) in 100 μL media containing 10% FBS, 25 mM glucose and 2 mM glutamine Cancer cells were treated with 6,8-bis-benzylthio-octanoic acid (CPI-613) and/or telaglenastat (CB-839, Cayman Chemical, Ann Arbor, Mich.), in RPMI media containing 5.8 mM glucose, 2 mM glutamine, 10% FBS and penicillin (100 IU/mL) and streptomycin (100 μg/mL) for 72 hrs. Conditioned media was removed and cells were allowed to recover for 24 hrs in RPMI media containing 25 mM glucose, 2 mM glutamine and penicillin (100 IU/mL) and streptomycin (100 μg/mL) without serum.

The cell-kill activity of CPI-613 and/or CB-839 was assayed at concentrations of 50-300 μM for CPI-613 and/or 1-75 μM for CB-839 along with a vehicle control. The number of viable cells was determined by using the CELLTITER-GLO® Assay (Promega, Inc., Fitchburg, Wis.). CELLTITER-GLO® reagent (60 μL per well) was added, and the cells were lysed for 5 minutes at room temperature, according to the instructions. The luminescence was measured using Filter Max F5 Micro plate reader (Molecular Device).

### Part II—Results

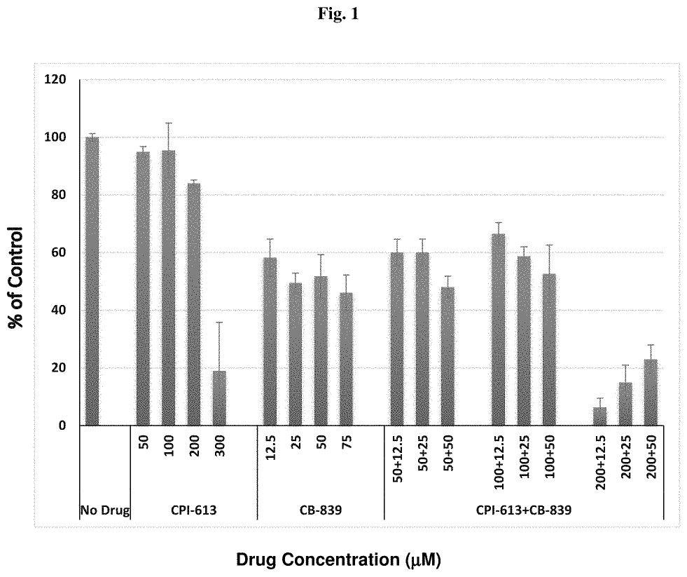

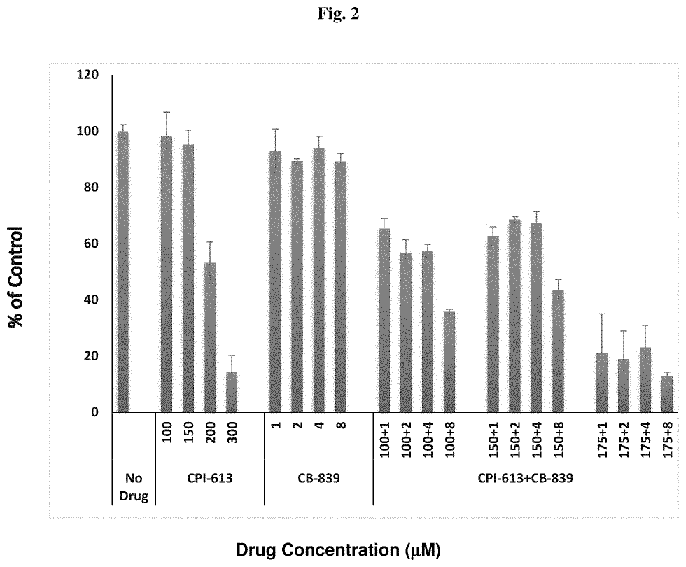

Results depicting the ability of CPI-613, CB-839, and combinations thereof to kill human pancreatic cancer AsPC-1 cells and human colon cancer LoVo cells are depicted in FIG. 1 and FIG. 2, respectively.

### Example 2—Activity of CPI-613, CB-839, and Combinations Thereof Towards Colon Cancer Cells In Vitro

The ability of CPI-613, CB-839, and combinations thereof to kill colon cancer cells may be evaluated according to the following protocol.

Human colon cancer (HT-29, COLO 205, and SW620) cell lines are obtained from American Type Cell Culture, Manassas, Va. All tumor cells are maintained at 37° C. in a humidified atmosphere with 5% CO₂ in T75 tissue culture flasks (except SW620), containing a) 10 mL of RPMI1640 (COLO 205), b) 10 mL of McCoy (HT-29), or c) 10 mL of L-15 media (SW620), each containing L-glutamine (2 mM), fetal bovine serum (FBS, 10%), penicillin (100 IU/mL) and streptomycin (100 μg/mL). SW620 cells are maintained at 37° C. in a non-CO₂ incubator. The cells are split at a ratio of 1:5 every 4-5 days by using trypsin and are re-suspended in fresh media in new flasks as described above. Cells are harvested for experiments at 70-90% confluence.

Cancer cells are seeded (5,000 per well in 96-well plate) in 100 μL media containing 10% FBS, 25 mM glucose and 2 mM glutamine Cancer cells are treated with 6,8-bis-benzylthio-octanoic acid (CPI-613) and/or telaglenastat (CB-839, Cayman Chemical, Ann Arbor, Mich.), in RPMI media containing 5.8 mM glucose, 2 mM glutamine, 10% FBS and penicillin (100 IU/mL) and streptomycin (100 μg/mL) for 72 hrs. Conditioned media is removed and cells are allowed to recover for 24 hrs in RPMI media containing 25 mM glucose, 2 mM glutamine and penicillin (100 IU/mL) and streptomycin (100 μg/mL) without serum.

The cell-kill activity of CPI-613 and/or CB-839 is assayed at concentrations of 50-300 μM for CPI-613 and/or 1-75 μM for CB-839 along with a vehicle control. The number of viable cells is determined using the CELLTITER-GLO® Assay (Promega, Inc., Fitchburg, Wis.). CELLTITER-GLO® reagent (60 μL per well) is added, and the cells are lysed for 5 minutes at room temperature, according to the instructions. The luminescence is measured using Filter Max F5 Micro plate reader (Molecular Device).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method for treating cancer in a human patient, comprising administering to the human patient in need thereof a therapeutically effective amount of (i) 6,8-bis-benzylthio-octanoic acid or a pharmaceutically acceptable salt thereof and (ii) a glutaminase inhibitor.

2. The method of claim 1, wherein the cancer is a lymphoma, leukemia, carcinoma, sarcoma, melanoma, myeloma, brain or spinal cord cancer, blastoma, germ cell tumor, cancer of the pancreas, colorectal cancer, myelodysplastic syndrome, or cancer of the prostate.

3. The method of claim 1, wherein the cancer is a lymphoma.

4. The method of claim 1, wherein the cancer is relapsed or refractory Hodgkin lymphoma.

5. The method of claim 4, wherein the patient has failed brentuximab vedotin and a PD-1 inhibitor.

6. The method of claim 1, wherein the cancer is relapsed or refractory T-cell non-Hodgkin lymphoma.

7. The method of claim 1, wherein the cancer is relapsed or refractory Burkitt's lymphoma.

8. The method of claim 1, wherein the cancer is high-grade B-cell lymphoma with rearrangements of MYC and BCL2 and/or BCL6.

9. The method of claim 1, wherein the cancer is a leukemia.

10. The method of claim 1, wherein the cancer is a carcinoma.

11. The method of claim 1, wherein the cancer is a sarcoma.

12. The method of claim 1, wherein the cancer is a myeloma.

13. The method of claim 1, wherein the cancer is a clear cell cancer.

14. The method of claim 1, wherein the cancer is a clear cell sarcoma.

15. The method of claim 1, wherein the cancer is a clear cell carcinoma.

16. The method of claim 1, wherein the cancer is a clear cell renal carcinoma.

17. The method of claim 1, wherein the cancer is a solid tumor.

18. The method of claim 1, wherein the cancer is a brain or spinal cord cancer.

19. The method of claim 1, wherein the cancer is a melanoma.

20. The method of claim 1, wherein the cancer is a blastoma.

21. The method of claim 1, wherein the cancer is a germ cell tumor.

22. The method of claim 1, wherein the cancer is a cancer of the pancreas.

23. The method of claim 1, wherein the cancer is a cancer of the prostate.

24. The method of claim 1, wherein the cancer is a myelodysplastic syndrome.

25. The method of claim 1, wherein the cancer is high risk myelodysplastic syndrome.

26. The method of claim 1, wherein the cancer is high risk myelodysplastic syndrome in a patient who has failed to respond, progressed, or relapsed while on hypomethylating therapy.

27. The method of any one of claims 1-26, wherein the glutaminase inhibitor is telaglenastat or a pharmaceutically acceptable salt thereof.

28. The method of any one of claims 1-26, wherein the glutaminase inhibitor is telaglenastat.

29. The method of any one of claims 1-26, wherein the glutaminase inhibitor is telaglenastat hydrochloride.

30. A pharmaceutical composition comprising (i) 6,8-bis-benzylthio-octanoic acid or a pharmaceutically acceptable salt thereof and (ii) a glutaminase inhibitor.

31. The pharmaceutical composition of claim 30, wherein the glutaminase inhibitor is telaglenastat or a pharmaceutically acceptable salt thereof.

32. The pharmaceutical composition of claim 30, wherein the glutaminase inhibitor is telaglenastat.

33. The pharmaceutical composition of claim 30, wherein the glutaminase inhibitor is telaglenastat hydrochloride.
